(19) United States
(12) Patent Application Publication
(10) Pub. No.: US 2002/0086022 A1
(43) Pub. Date: Jul. 4, 2002

(54) Neutralizing antibody and immunomodulatory enhancing compositions

(76) Inventor: Gary R. Davis, Tulsa, OK (US)

Correspondence Address: MUNSCH, HARDT, KOPF & HARR, P.C., INTELLECTUAL PROPERTY DOCKET CLERK, 1445 ROSS AVENUE, SUITE 4000, DALLAS, TX, 75202-2790, US

(21) Appl. No.: 09/780,773

(22) Filed: Feb. 9, 2001

Related U.S. Application Data
(60) Provisional application No. 60/182,428, filed on Feb. 14, 2000.

(51) Int. Cl. A61K 39/42; C12P 21/04; C07K 16/10

(52) U.S. Cl. 424/160.1; 530/389.4; 435/70.21

(57) ABSTRACT

A composition and method for immune modulation of pathogenic infections is disclosed. More particularly, a composition and method for enhancing a mammal's ability to respond to, e.g., immunosuppressive pathogens, is disclosed in which a target antigen is mixed with a heterologous antisera specific or cross-reactive to that antigen to produce an inoculant. The inoculant is used to cause natural and specific immune responses to the heterologous antibodies to enhance complement fixation and specific humoral and cellular responses.

### TECHNICAL FILED OF INVENTION

This invention in the field of cell biology, physiology and medicine relates to the bioactive compositions containing a combination of antigen and heterologous antibodies to produce an immunomodulatory composition for use in treating immunosuppressive infections.

### BACKGROUND OF THE INVENTION

Without limiting the scope of the invention, its background is described in connection with immunosuppressive viruses, e.g., SIV and HIV, as an example.

Acquired immune deficiency (AIDS) is recognized as an epidemic in several areas of the world, including the United States. Human Immunodeficiency Virus (HIV-1), a retrovirus, has been identified as the etiologic cause of the disease. HIV-1 was previously identified as Human T-Cell Lymphotropic Virus Type III (HTLV-III and Lymphadenopathy Associated Virus (LAV). To date, two related but distinct viruses HIV-2 and HIV-3, have been identified. HIV-2 is closely related to Simian Immunodeficiency Virus (SIV-mac), which causes an AIDS-like disease in macaques. Alignments of the nucleotide sequences of HIV-2 and SIV reveal a considerable homology between HIV-2 and SIC-mac. These two viruses share approximately 75% overall nucleotide sequence homology, but both of them are only distantly related to HIV-1 with approximately 40% homology. Sub-strains of HIV-1, HIV-2 and HIV-3 have been identified. All strains and sub-strains may be referred to individually, HIV-1, HIV-2 and HIV-3 have been identified. All strains and sub-strains may be referred to individually, HIV-1, HIV-2 and HIV-3 and their sub-strains may cause immune suppression in humans.

The groups at high risk of infection with HIV include homosexual and bisexual men and abusers of injected drugs. Before the advent of reagents that made large-scale screening for HIV antibodies available, high-risk groups included whole blood transfusion recipients. Other predictable high-risk groups are women artificially inseminated with sperm from infected donors, sexual partners of those infected with HIV, recipients of organs and fetuses in HIV infected women. Evidence indicates that HIV is transmitted heterosexually.

Presently, the therapies in use are generally limited to regimens designed to treat the opportunistic infections and neoplasias associated with AIDS and its related illnesses and to target the replicative cycle of the virus. Very few treatments are available, however, which attach HIV, the underlying cause of AIDS, a fatal disease. Among the known antiviral and antiretroviral drugs, which are believed to merely slow down viral replication and that do not cure the disease, are azidothymidine (AZT), alpha interferon, gamma interferon, azimexon and isopinosine.

Remission of some Karposi's Sarcomas have been reported following treatment with alpha interferon, but other antiviral drugs have not proven effective against HIV infections. Immunomodulators, such as cimetidine and interleukin-2, which are intended to stimulate T-cells and natural killer cell activity, have also been reported as useful in the treatment of AIDS. Similar claims have also been made in connection with indomethacin, an anti-inflammatory and prostaglandin inhibitor. In summary, current methods for treating individuals infected with HIV are few, and largely ineffective.

In addition to HIV, a number of infectious diseases are caused by virulent immunosuppressive pathogenic organisms. These disease states are often accompanied by other opportunistic infections and/or diseases due to the compromised immune system of affected patients.

There is new evidence that new epidemics are emerging throughout the industrialized, developing and transitional countries of the world. For example, in South Africa alone, the harm caused by the HIV in confirmed by the finding that more than 14% of its nearly 40 million citizens are infected with HIV. By 2010, the national infection rate in South Africa is expected to reach 25%. In other parts of the world, the life expectancy of the adult population is expected to drop 10 to 15 years by the year 2010. The human devastation, pain, suffering and ultimately death to victims are occurring at rates unmatched in the history of man.

Today, the rates of reported cases of HIV infections are increasing in geometric proportions and clinical treatments represent marginal improvements in the management of health care in this area. The rapid spread of HIV infection is out of control. Unless improved treatments are found, the future outlook for the state of the world's health is dismal.

In addition to the genes that encode structural proteins (the virion capsid and envelope glycoproteins) and the enzymes required for proviral synthesis and integration common to all retroviruses, HIV and SIV encode genes that regulate virus replication as well as genes that encode proteins of yet unknown function. The only notable difference in the genetic organizations of HIV and SIV resides in the open reading frame referred to as vpx, which is absent in HIV-1 and vpu in HIV-1 but not in HIV-2 and SIV. These viruses are both tropic and cytopathic for CD4 positive T-lymphocytes. A great number of studies have indicated that CD4 cells function as an incubator for viral replication.

Some therapeutic success has been observed following intravenous immunoglobulin treatment of HIV-infected children (Clavelli, et al., Padiatr. Infec. Dis. 5SS207 (1986)). It has been proposed that this treatment may be particularly beneficial to HIV-infected children. These children exhibit increased susceptibility to bacterial and viral infections due to both the destructive effects of HIV-1 infection and because infants possess an immature immune system. Specific anti-HIV-1 antibodies may have protective effects against infection. Passive administration of immunoglobulin from asymptomatic, HIV-1 positive individuals has led to a temporary clinical improvement in these individuals (Wendler, et al, AIDS Res. And Hum. Retroviruses 3:177 (1987) and Rank, et al., Clin. Exp. Immunol. 69:231 (1987)). Another study has shown that children born to HIV-1 positive mothers were less likely to be infected with HIV-1 if they possessed serum with high neutralizing activity (Broliden, et al., AIDS 3:577 (1989)).

These studies indicate that the presence of maternal antibodies may confer protection when passed from mother to child. Transfusion of serum from HIV-1 infected individuals, however, is not feasible on a large scale from a logistical view. Furthermore, it is unlikely to have broad application as a number of sub-strains have been identified that would evade immune surveillance.

One source of protective immunoglobulin to HIV-1 infected patients is from already infected individuals. The use of human antisera from infected individuals carries with it the obvious risk of additional infection to infected patient and health care workers. In addition, such immunoglobulin may contain virus particles which could be infectious to treated populations, thus complicating antisera production if not ultimately patient therapy.

### SUMMARY OF THE INVENTION

The subject invention relates, in one aspect, to pharmaceutical composition containing as active ingredients thereof (1) heterologous antibodies reactive with a pathogenic organism, such as a virus, bacteria, fungus, venom, pollen and the like, to which the antibodies are specific or cross-reactive; and (2) the antigen to which the heterologous antigens are specific or to which they cross-react. The reactive or cross-reactive antibodies may be polyclonal or monoclonal. The present invention is based on the recognition that natural antibodies react or cross-react with heterologous antibodies. By heterologous antibodies, it is meant that the antibodies are derived from a different source than the host or target immune system.

As an illustration of the production of pathogenic reactive antibodies, anti-HIV polyclonal serum is produced by immunizing an animal with HIV viral lysate, i.e., purified or semi-purified components of live or inactivated virus. Alternatively, DNA isolated from HIV virus may be used to produce HIV antigen by recombinant methods. A variety of animals can be immunized with such a lysate including mice, rabbits, horses, cows, donkeys, sheep, pigs, humans, monkeys and primates (including humans) to produce the heterologous antibodies. When crossing species, the term heterologous is used as the antibodies are from a different source. The passive transfer of xenogenic antibodies has long been used in the case of Rh disparate mother and child in the form of RhoGam®. The problem with xenogenic antibody transfer, however, is that the host may develop a strong immune response to the transferred antibodies.

Historically, it was specifically recognized that in patients with normal immune systems, it would be counter-indicated to immunize with large foreign proteins, such as xenogenic antibody-antigen complexes. In most cases, it would be counter-indicated to provide passive immunity with xenogenic antibodies, as a strong immune response would normally be mounted against those antibodies. However, because of similarities between the goat immune system and human immune system, the inoculation of a human with a normal immune system with goat antibodies will not result in the severe immune complex reactions customarily anticipated with other foreign animal proteins. The present invention is based on the recognition that the immune-suppressed patients that are to receive the inoculation of the present invention do not have normal immune systems. In fact, the compositions and method of the present invention are designed to activate the host immune system by activating natural and acquired immune responses.

Humans, other primates and goats may be used to produce antibodies for use with the present invention, with goats being a particularly useful animal for this purpose. The discussion that follows focuses on the goat. The antibody ingredient present in the bioactive compositions of this invention may be of male or female goat origin. In one embodiment of this invention, anti-HIV-1 antibodies are produced by administering to a goat HIV-1 encoded protein in an amount sufficient to stimulate an immune response. The HIV-1 encoded protein may be purified from a lysate of HIV-1 infected cells or it may be produced by recombinant methods.

The goat antibodies produced as described herein may be formulated in accordance with this invention in a composition for inhibiting viral replication in vitro or in vivo biologic system. The inhibitory effectiveness reasonably suggests its administration as an immunotherapeutic to humans infected by the HIV-1 virus, or related viruses.

More particularly, the present invention is directed to an immunostimulatory composition or vaccine for stimulating immune responses in an immunosuppressed host that includes heterologous antibodies specific for an antigen. The heterologous antibodies form a complex with the antigen. Also, the heterologous antibody-antigen complex may be mixed with a pharmaceutically acceptable carrier. In particular, the bases of the immunostimulatory composition is based on the realization that current vaccines and therapies fail to address the immunosuppression of the individuals affected by an immunosuppressive pathogen. In fact, the present invention is based on the counter-intuitive recognition that the immunosuppressed patient is unable to mount a specific immune response using T cell mediated immunity. By providing the specific antigen that is causing the infectious disease in a killed or attenuated form with heterologous antibodies, the immune system of the host is able to recognize the heterologous antibodies and mount an immune response that coordinated both the anti-antibody and the anti-antigen response of the host.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a graph depicting the inhibition of SIV infection expressed as the number of fusion sites in CEMx174 cells by various dilutions of goat anti-SIV serum over time;

FIG. 2 is a graph depicting the inhibition of SIV infection expressed as the number of fusion sites in CEMx174 cells by various dilutions of goat anti-SIV serum at Day 2 post-infection versus a normal serum control; and

FIG. 3 is a graph depicting the inhibition of SIV infection expressed as the number of fusion sites in CEMx174 cells by 1:20 and 1:80 dilutions of goat anti-SIV serum over time.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

### Heterologous Cross-Reactive Antibody Activity

An immunocompetent animal will produce an immunological response to a foreign protein. The immunological response to the foreign protein predictably comprises both humoral (antibody) and cellular (lymphocyte and phagocyte) immune factors. The humoral response produces antibodies which include, but are not limited to, IgM, IgG and IgA. The antibodies produced may be either of two classes: (a) a neutralizing antibody, i.e., an antibody not requiring the complement system for cellular or viral destruction; and (b) a complement fixing antibody.

The practice of using vaccinations as a technique for acquiring immunity has occurred since at least the 1700's when Edward Jenner first recognized the correlation between cowpox and the lessening of the virological virulence of smallpox in milkmaid's population.

In this historical event, in 1798, Jenner inoculated a live cowpox virus into an immunologically healthy boy, and upon subsequent inoculation of the child with live smallpox virus, the child did not develop life-threatening smallpox. In this instance, the outer membrane of the cowpox virus was so similar to the smallpox virus that the body's immune system could not tell the two apart; thus, antibodies raised against cowpox virus administered to a human also could react against invading smallpox virus. This method required the direct administration of a virus to humans, said virus being capable of causing disease in bovines but not in humans.

It has now been found that viral neutralization or bacterial lysis by antibodies developed in one mammal upon exposure to a virus or bacteria, respectively, can be administered to another mammal to provide treatment for the suppression or prevention of infections caused by direct exposure to the virus or bacteria.

Higher animals have, by evolution, established several very effective means of defense against microbes involving the immune system. Invading bacteria are rapidly identified, via complement and immunoglobulin opsonization, phagocytozed and destroyed by the cellular immune system and white blood cells (neutrophils and macrophges). Globulins are essentially nature's perfect antibodies. Complement, available as a precursor protein which is activated by the presence of microorganism and globulins, also function in antibacterial activities. Opsonization of foreign organisms in the memory component of the immune system. After previous antigenic exposure, the immune system produces a series of globulins which attach to and coat bacterial or neutralize viruses so that they are readily recognized, phagocytosed and destroyed by neutrophils and macrophages. Foreign proteins of invading organisms also stimulate a homoral immune response which over a period of time (3-6 weeks) amplifies the number of cells designed to recognize and destroy specific invaders. Tables 1 and 2 present the antimicrobial functions of immunoglobulins and the metabolic properties of immunoglobulins.

TABLE 1
ANTIMICROBIAL FUNCTIONS

| | |
|---|---|
| 1 | Bacterial lysis (requires complement) |
| 2 | Opsonization (enhanced by complement) |
| 3 | Toxin neutralization |
| 4 | Viral neutralization (may be enhanced by complement) |
| 5 | Mediates Antibody Dependent Cell-Mediated Cytoxicity (ADCC) |
| 6 | Synergistic activity with antibiotics |

TABLE 2
METABOLIC PROPERTIES OF IMMUNOGLOBULINS

| | IgG | IgA | IgM | IgD | IgE |
|---|---|---|---|---|---|
| Serum level | 989 | 200 | 100 | 3 | 0.008 |
| Means (mg/dl) (range) | (600-1600) | (60-300) | (45-150) | | |
| Total body pool | 1030 | 210 | 36 | 1.1 | 0.01 |
| Synthesis rate | 36 | 28 | 2.2 | 0.4 | 0.004 |
| Plasma half life | 21 | 5.9 | 5.1 | 2.8 | 2.4 |
| Fractional turnover rate | 6.9 | 24.0 | 10.6 | 37.0 | 72.0 |
| Fraction for each class in plasma means | 0.52 | 0.55 | 0.74 | 0.75 | 0.51 |

The fraction represents the portion of the total immunoglobulins of each class that is found in the plasma of humans.

Host responses are initiated only after foreign substances, such as bacterial, fungus, protozoa, parasites or viruses which already have colonized tissues and are beginning to enhance their own defenses (antigen masking, replication, biofilm, toxins). The host defense strategies require time to reach peak responses. During this time period, serious infection may be established, especially in immuno-compromised patients. The presence of tissue damage and foreign bodies lower thresholds of infection and diminishes effective responses, thus, the foreign agent is neutralized or coated by antibodies concurrently. The complementary activity would synergistically optimize the antipathogenic effectiveness of the bioactive compositions of this invention.

The bacteria contemplated within the scope of this invention includes Salmonella typhi, Shigella sonnei, Shigella Flexneri, Shigella dysenteriae, Shigella boydii, Eschericia coli, vibrio cholera, Group D-2, Group E, Group G, Group I, Group 1, Listeria, Erysipelothrix, Mycobaterium, Aerobic pathogenic Actinomycetales, Enterobacteriaceae Vibrio, Pseudomonas, Plesiomonas, Helicobacter, W. succinogenes, Acineto bacter spp., Flavobacterium, Pseudomonas, Legionella, Brucella, Haemophilus, Bordetalla, Mycoplasmas, Gardnerella, Streptobacillus, Spirillum, Calymmatobacterium, Clostridium, Treponema, Borrelia, Leptospira, Anaerobic Gram-negative Bacterial including bacilli and Cocci, Anaerobic gram-Positive Nonsporeforming-bacilli and Cocci, Yersinia, Staphylococcus, Clostridium, Enteroccus, Streptococcus, Aerococcus, Planococcus, Stomatococcus, Micrococcus, Lactococcus, Gernella, Pedioccouccus, Leuconostoc, Bacillus, Neisseria, Branhamella, Coryne bacterium, Campylobacter, Arcancbaterium haemolyticum, Rhodococcus spp., Rhodococcus, Group A-4.

Also included are resistant or non-resistant bacteria selected from the group consisting essentially of Enterobacteriaceae, Klebsiella sp., Bacteroides sp., Enterococci, Proteus sp., Streptococcus sp., Staphylococcus sp., Pseudomonas sp., Neisseria sp., Pedptostreptococcus sp., Fusobaterium sp., Actinomyces sp., Mycobacterium sp., Listeria sp., Corynebacterium sp., Proprionibacterium sp., Actinobacillus sp., Aerobacter sp., Borrelia., Campylobacter sp., cytophaga sp., Pasteurella sp., Clostridium sp., Enterobacter aerogenes, Peptococcus sp., Proteus vulgaris, Proteus morganii, Staphylococcus aureus, Streptococcus pyogenes, Actinomyces sp., Campylobacter fetus, and Legionella pneuophila, ampicillin-resistant strain of S. aureus, and methicillien-resistaant strain of S. aureus.

The viruses contemplated within the scope of this invention include hepatitis A, hepatitis B, hepatitis C, Varicella-Zoster virus, Rotaviruses, polio virus, human immunodeficiency virus (HIV), herpes simplex virus type 1, human retroviruses, herpes simplex type 2, Ebola virus, cytomegalo viruses, Herpes Simplex viruses, Human cytomegalovirus, Varicella-Zoster Virus, Poxvirus, Influenza viruses, Parainfluenze viruses, Respiratory Syncytial Virus, Rhinoviruses, Coronaviruses, Adenoviruses, Measles virus, Mumps virus, Rubella Virus, Human Parvoviruses, Arboviruses, Rabies virus, Enteroviruses, reoviruses, viruses Causing gastroenteritis Hepatitis Viruses, Filoviruses, Arenaviruses, Papillomaviruses, Polymaviruses, Human Immonodeficiency viruses, Human Retroviruses, Spongiform Encephalopathies, Amyotropic Lacteral Sclerosis, and Multiple Sclerosis.

In the last decade, intravenous immunoglobulins (IVIG) have become a major treatment regime for bacterial and viral infections and of primary and secondary immunodeficiency states. For example, Bucklye, et al., New Eng. J. Med. 325:110-117 (1991), describe using intravenous immune globulin in the treatment of immunodeficiency diseases, and Cometta, et al., New Eng. J. Med. 327:234-239 (1992), describe the prophylactic intravenous administration of standard immune globulin and core lipopolysaccharide immune globulin in patients at high risk of post-surgical infection. IVIGs are prepared from the pooled plasmas of large numbers of donors, and tend to have a broad representation of antibodies. Specifically, pooled polyvalent human globulins usually contain antibodies for ubiquitous pathogens such as H. Influenza type b, pneumococci, staphylococci, diphtheria, tetanus, respiratory syncitial virus (RSV), measles, cytomegalovirus (CMV), and varicella zoster virus. Antibody concentrations from lot to lot and from manufacturer to manufacturer usually vary only two to four fold when measured by anitbody binding assays. However, functional assays often show much larger lot to lot variations as do antibody concentrations to less common pathogens (see, Siber, et al., “Use of immune globulins in the prevention and treatment of infections”, Current Clinical Topics in Infectious Disease, Remington J S, Swartz MM, eds., Blackwell Scientific, Boston, 12:208-257 (1992).

IVIG therapy has been reported to be beneficial for more than thirty-five diseases produced by immunopathologic mechanisms. Passive immunization depends on the presence of high and consistent titers of antibodies to the respective pathogens in each preparation.

Nosocomial infections are derived from the hospital or clinical setting, and are also a serious problem. Specifically, bacteria and viruses present in the hospital or clinic can infect a recovering patient and put the patient at risk or prolong the recovery period. A patient's risk factors for nosocomial infection can be intrinsic, such as susceptibility to infection due to immunosuppression, or extrinsic, such as invasive medical interventions (e.g., surgery or use of medical devices such as catheters, ventilators, etc.).

Staphylococcus aureus is an important cause of nosocomial infection, especially nosocomial pneumonia, surgical wound infection and bloodstream infections (Panlilio, et al., Infect. Cont. Hosp. Epidemiol. 13:582-586 (1992). Other pathogens commonly associated with nosocomial infection include, but are not limited to, Escherichia coli, Pseudomonas aeruginosa, Enterococcus supp., Enterobacter supp., coagulase-negative staphylococci (CNS), and Candida albicans (Emori, et al., Am. J. Med. 91: (Suppl 3B) 289S-293S (1991). Hospitals and clinics typically employ strict sterilization procedures and use antibiotics such as methicillin, oxacillin, and nafcillinio combat virulent bacterial pathogens. However, nosocomial infections still occur in great numbers and are expected to increase with a aging population.

The use of intravenous immunoglobulins to prevent nosocomial infections has been discussed in Siber, New Eng. J. Med. 327:269-271 (1992). Passive immunization against infections has been particularly successful using immune globulins containing antibodies specific for tetanus, hepatitis B, rabies, chickenpox, and CMV. However, it is reported that there is an inconsistent benefit from using intravenous immune globulins to prevent nosocomial infections. This may be due to variable lot-to-lot levels of antibodies to the more common nosocomial pathogens and emerging new serotypes.

U.S. Pat. No. 4,412,990 to Lundblad, et al. Discloses an intravenous pharmaceutical composition containing immunoglobulin (IgG) and fibronectin that exhibits a synergistic opsonic activity which results in enhanced phagocytosis of bacterial, immune complexes and viruses.

U.S. Pat. No. 4,994,269 to Collins, et al. discloses the topical use of monoclonal antibodies for the prevention and treatment of experimental P. aeruginosa lung infections. Specifically, the antibodies are administered via aerosol spray to the lungs. Results show beneficial effects in the treatment of Pseudomonas pneumonia.

U.S. Pat. No. 4,714,612 to Nakamura, et al. discloses the use of a non-specific gamma globulin IgG in a mouthwash for preventing gingivitis. Ma, et al., Arch. Oral Biol., 35 suppl: 115s-122s, 1990, discloses the use of monoclonal antibodies specific for Streptococcus mutans in a mouthwash. Experiments showed control subjects experienced recolonization with Streptococcus mutans within two days, but those treated with the monoclonal antibodies remained free of Streptococcus mutans for up to two years.

The present invention provides for the direct, concentrated, injected or transfusion delivery of passive immunity to a specific antigen and redirection of the host immune system to the antigenic entity. The present invention provides new compositions that include a full repertoire of immunoglobulin classes (IgG, IgA, IgM) and methods for prophylactic positioning of the compositions wherein the compositions are applied directly to stored blood (banked blood) wounds, burns, latex, rubber, tissues (including the inner and outer surface of the skin body cavities, mouth, system, etc.) and biomaterial devices as a cream, gel, ointment, vaginal and rectal products, coating layer, or the like to prevent and treat infections from microorganisms and viruses.

The infection may also be provided as a biocompatible vaccine by providing the heterologous immunoglobulin-antigen complex in a biodegradable matrix (such as those that are poly lactic acid based) to provide a broad spectrum of antibodies to specific infectious pathogens immobilized thereon or encapsulated therein. The invention facilitates the predetermined timed-release of bioactive compositions of this invention in the treatment of and for the prevention or substantial inhibition of pathogenic replication.

The invention may also be used to provide a method of using the immunomodulatory entrapped antigens and heterologous immunoglobulin compositions in high concentration, whereby retroviruses or other immunosuppressive pathogenic organisms (virus, fungus, bacteria and/or protozoa and the like) cause opportunistic infections are pre-opsonized in situ for enhanced phagocytosis and killings.

According to the invention, the direct, concentrated local delivery of passive immunity is accomplished by applying a composition having a full repertoire of immunoglobulins (IgG, IgM and IgA) to biomaterials, implants, tissues and wound and burn sites. The compositions preferably have elevated concentrations of certain immunoglobulin classes (IgG, IgM and IgA), and elevated antibody titers to specific microorganisms that commonly cause biomaterial, burn, mucosal, tissue, surgical wound and body cavity infections. Compositions within the practice of this invention may take several forms, including cremes, gels, ointments, lavage fluids, sprays, lozenges, coatings, layers or any of the topical mode of administration. In addition, the compositions may be combined with or immobilized on a biocompatible and/or biodegradable material, or be impregnated in or encapsulated within a biocompatible-biodegradable polymeric matrix material for sustained release. The compositions can be used for both prevention and treatment of infections.

In oral applications, the compositions would ideally be provided as a lozenge, mouthwash or spray, while, in trauma patients, the composition may be best applied as a cream or ointment, or as part of a fluid infusion, biomaterial implant or fixation device. The immunoglobulins and other antibodies of the present composition can be immobilized on a biocompatible and/or biodegradable material or encapsulated within a biodegradable-biocompatible matrix/microspheres which is placed in-situ in a patient's infected area, wound site or surgical area, or to be coated on a catheter or the like that is inserted in a body cavity.

Where the inhibition of immunosuppressive pathogenic organisms is substantial, the pathogen load (i.e. viral, bacterial) will be reduced and the potential for the production of toxins by bacterial, virus, fungus or other microbes minimized.

Upon creating humoral and cellular-immunity in a non-human mammal, introduction of the resulting IgA, IgGI, and/or other neutralizing antibodies and other immunocompetent antibodies and cellular activity on or into a system containing a virus, such as human immunodeficiency virus (HIV), prevents virus replication and renders that virus noninfectious. In so doing, further viral replication may be retarded, blocked or stopped by means of the IgA, IgGI, other neutralizing antibodies and cellular immune action.

An immunocompetent animal makes antibodies in response to simian immunodeficiency virus (SIV), HIV and/or other viruses. These antibodies react with the virus in an antigen-antibody reaction. The normal response for antibody production is for IgM to be produced first, followed by IgG. The antibodies IgM and IgG are not capable of activating or causing biodestruction of some viruses, such as the SIV or HIV virus. It is believed that the bases for this incompetent antigen-antibody response is that the SIV/HIV virus either produces intrinsically, through either viral synthesis or host cell synthesis, an SIV, HIV or viral complement inhibiting factor which prevents activation of the complement system, i.e., viral destruction. The incompetent antigen-antibody response is itself incapable of activating the body's complement system, because the antibodies IgM and IgG are known to be complement fixing antibodies. Thus, where the incompetent antigen-antibody response is detected by standard techniques, the incompetent antigen-antibody response can be eliminated or bypassed as an inhibiting factor by the use of antibodies that are of the neutralizing antibody class.

Using this methodology of inserting into mammals viruses for which IgM and IgG may not activate or cause biodestruction such as SIV, HIV, Polioviruses, Influenza, Hanta virus, pox viruses, Caprine Encephalitis (CAE) virus, Herpes-viruses, Hepatitis, Encephalitis, measles, mumps, Ebola, and/or Rubella, serum containing neutralizing antibodies and cellular immunity may be obtained naturally. These products when suitably treated and prepared may yield as a minimum the following: (a) a vaccine suitable for use in humans; (b) an immunological barrier to virus transmission via mucosal surfaces by way of creams, sprays, liquids and swabs; and (c) a serum to arrest further viral development.

Each of these applications for inhibiting or preventing the proliferation of abnormal cells in biological systems (in vitro or in vivo) is based upon, but not limited to, the use of antibodies produced in non-human mammals in a patient or subject which, upon being exposed to SIV, HIV or similar viruses, does not die but rather produces an immunological reaction. The mammal's immunlogical reaction to the introduction of the SIV and/or HIV virus yields antibodies which are complement fixing and/or neutralizing antibodies and also cellular immune reactions which when treated in accord with accepted procedures yield the production of a usable serum immunoglobulin which can then be used to prevent further virus replication.

It is known that, in procedures described herein, SIV, HIV and their immunoglobulin, in particular, IgA, do not affect non-human hosts. Riott, Ivan, Essential Immunology, Blackwell Scientific Publications, Cambridge, MA (1991). A non-human host that may be used to demonstrate and develop serum or milk containing neutralizing antibodies and cellular immunity is and advanced pregnant female goat. The goat was chosen because it does not die from infection with SIV or HIV and a pregnant female produces large quantities of neutralizing antibodies that may be isolated from serum and/or milk. Neither a pregnant female goat, nor a female goat, is required. A pregnant female goat is preferred in order to provide a higher level of production as an additional source of IgA, IgGI and other neutralizing antibodies. Other non-human mammals may also be used to obtain the neutralizing antibodies of interest provided that, when exposed to SIV, HIV or similar viruses, they do not die but rather produce humoral and cellular reactions. Although not required, the non-human mammal's immunological reaction can also be potentiated by use of an appropriate adjuvant.

The neutralizing antibody may be administered to humans either intravenously, intradermally, subcutaneously, intramuscularly or as a vaccine prepared from serum or milk. For persons with immature digestive systems which are capable of absorbing antibodies or antibody-making cells, such as newborn infants, the neutralizing antibodies may also be ingested via milk or a modified milk product.

The simian immunodeficiency viruses (SIVs) were originally isolated from rhesus monkeys (Macaca mulatta) with immunodeficiency or lymphoma (SIVmac). Subsequently, SIVs were isolated from asymptomatic mangbey monkeys (SIV/ssm, SIV/smlv, and SIV/Delta) and from Macaca nemestrina with lymphoma (SIV/Mne). A strain of SIV thought to be obtained from African green monkeys has since been shown to be SIVmac. Recently, an authentic SIVagm virus was isolated from naturally infected African green monkeys.

In susceptible primate species, SIVs cause a fatal disease which symptoms similar to those associated with the human acquired immunodeficiency syndrome (AIDS) caused by human immunodeficiency viruses type 1 (HIV-1) or type 2 (HIV-2) or type 3 (HIV-3). The genomic organizations of HIV-1, HIV-2, HIV-3 and SIVmac or SIVagm are very similar, and there is approximately 40% sequence relatedness between SIVmac and HIV- 1 or SIVagm and an even closer relatedness 65% overall sequence identity between SIVmac and HIV-2. Because of these similarities between HIV and SIV, SIV macaque model is suitable for the development and testing of bioactive agents against human AIDS in vitro.

### EXAMPLE I

### Preparation of Concentrated Simian Immunodeficiency Virus (SIV)

A macrophage-tropic strain of SIV (SIVmac239-17E) referred to as “SIV-17E” was prepared by growing the virus in CEMx174 cells. Sharma, et al., “Derivation of neurotropic lymphocytotropic parental virus: pathogenesis of infection in macaques,” J Virol 66:352-3556 (1992). The CEMx174 cell is an immortalized CD4-bearing human T/B hybrid cell line that is highly susceptible to SIV-induced cytophaticity (fusion) and permissive for replication by SIVmac. Hoxie, et al., “Biological characterization of a simian immunodeficiency virus-like retrovirus (HTLV-IV): Evidence for CD4-associated molecules required for infection,” J. Virol 62:2557-2568 (1988). Koenig, et al., “Selective infection of human CD4 +cells by simian immunodeficiency virus: productive infection associated with envelope glycoprotein induced fusion, ” Proc. Nat'l Acad. Sci. USA 86:2443-2447 (1989).

The cells were grown in RPMI-1640 medium (“RPMI'I) supplemented with 10 & fetal bovine serum, glutamine and gentamicin, and were used for preparation of stock virus and the virus neutralization assay. Cell cultures (9 milliliter) were inoculated with 1 milliliter of virus (10⁴ TCID 50/milliliter) and examined for cell fusion. When approximately 50% of the cells had been used, the cultures were expanded by the addition of fresh cells. Cultures were further monitored for infectivity by fusion and reverse transcriptase. Supernatant fluids (approximately 240 milliliters) were collected and clarified by centrifugation. The stock contained 10 4 TCID 5 50/milliliter in CEMx174 cells. Virus was pelleted at 27,000 rpm in a SW28 rotor (Beckman Instruments, Inc., Fullerton, CA) for two hours at 4 degrees Centigrade, resuspended in two milliliters NET buffer (50 mM HC1, 5 nM ethylenediaminetetraacetic acid, 10 mM Tris hydrochloride, pH 7.4) and purified on a Sepharose CL-48 column (Pharmacia Diagnostics, Inc., Fairfield, NJ).

### EXAMPLE II

### Neutralization of the SIV Virus in Vitro

Neutralizing antibody or antibodies obtained from a non-human mammal exposed to SIV virus were examined for antiviral activity in vitro. Simian immunodeficiency virus (SIV) was prepared according to procedures given in Example I. The virus was heat-killed in a 60 deg C water bath for thirty minutes. Killed virus was used for the sake of safety; however, use of live virus will result in a faster immunogenic response.

A pregnant female goat was exposed to the simian virus by intramuscular injection. The goat was injected with a one milliliter suspension of killed SIV at 1 X 10 6 viral particles per milliliter once per week for three weeks. The goat's immunogenic response was augmented using the MPL(R) (RIBI IMMUNOCHEM RESEARCH, INC.) +TDM Adjuvant System (Sigma Chemical Co., St. Louis, MO) according to the manufacturer's instructions, i.e., administering intramuscularly 500 ul into each hind leg.

Following exposure of the goat to simian SIV virus, the animal's serum and/or milk was obtained. Milk was obtained from the goat as soon as it became available, generally at about three weeks after the birth of the kid. The mild was frozen until subsequent use.

To collect serum, blood samples of at least 10 cc were drawn from a large bore vein after the first week and weekly thereafter for twelve weeks. This regimen was selected to optimize the opportunity to first detect neutralizing antibodies. Each serum sample was obtained by centrifugation of the blood sample, frozen for transport to the laboratory, and subsequently tested for neutralizing antibodies. The neutralizing antibodies were first detected on or about the eighth week. During subsequent weeks, samples were tested to monitor changes in neutralizing antibody concentration, and the amount of antibodies detected increased over time. Straight, untreated serum (without concentrating the antibodies) obtained from the blood sample taken at the twelfth week was used in the in vitro neutralization studies.

A neutralization assay was performed to demonstrate the ability of the neutralizing antibodies and cellular immunity in the goat serum to prevent infectivity of the SIV virus in vitro. SIV-17E virus (100 TCID So/milliliter) was incubated with doubling dilutions of the goat serum at 37 deg C for one hour. In a 96-well tissue culture plate 100 microliters of each SIV-17E/goat serum mixture was added to wells using three wells per dilution. Approximately 5 x10⁴ CEM174 cells were then added to each well. The cultures were incubated at 37 degrees Centigrade and observed for fusion over a period of five days. Fusion was observed in control cultures within one day. The neutralizing antibody titer was taken as the highest dilution of serum which prevented cell fusion.

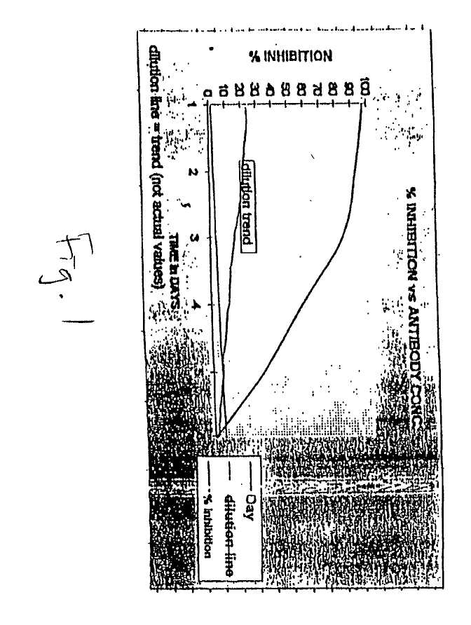

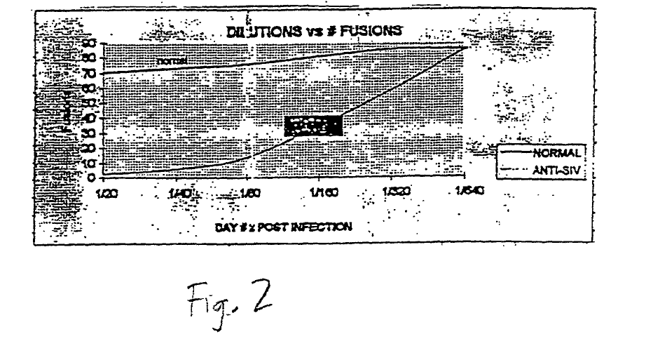

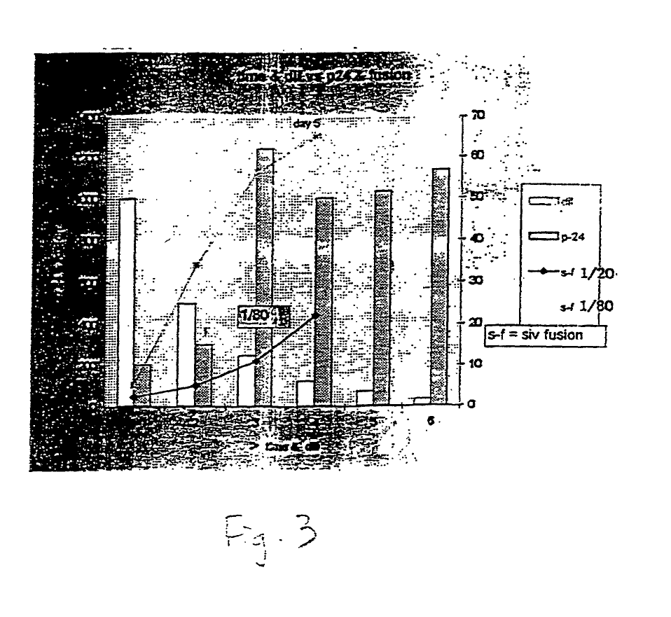

The results of the neutralization assay are given in Table 3 and illustrated in FIG. 1 is the average number of fusion sites observed over time for various dilutions of goat anti-SIV serum (Go-SIV), compared to normal goat serum (NGS) as the control. FIG. 2 present neutralization data for Day 2 post-infection. FIG. 3 depicts neutralization of SIV infection over time by goat anti-SIV serum used at a 1/20 and a 1/80 dilution. Table 4 presents the neutralization assay data as a percent of inhibition of SIV fusion sites by various dilutions of goat anti-SIV serum at day 2 post-infection.

A 1/20 dilution of the goat anti-SIV serum almost completely inhibited SIV infection of the CEM174 cells (97.2% inhibition). Dilutions of 1/40 and 1/80 inhibited 92.4% and 83.7%, respectively. These results indicate that the grant anti-SIV serum contains potent neutralizing antibodies which can be used to block the infectivity of the SIV virus.

TABLE 3
AVERAGE NUMBER OF FUSION SITES OVER TIME FOR
VARIOUS DILUTIONS OF GOAT ANTI-SIV SERUM
vs.
THE NORMAL SALINE CONTROL

| DIL | NORMAL | ANTI-SIV |
|---|---|---|
| 1/20 | 70 | 2 |
| 1/40 | 72.5 | 5.5 |
| 1/80 | 75 | 13 |
| 1/160 | 80 | 34 |
| 1/320 | 85 | 58 |
| 1/640 | 85 | 85 |

TABLE 4
PERCENT INHIBITION OF SIV FUSION SITES BY DIFFERENT
DILUTIONS OF GOAT ANTI-SIV SERUM (Day 2-post-infection)

| | DILUTION | INHIBITION |
|---|---|---|
| 1 | 1/20 | 97.2 |
| 2 | 1/40 | 92.4 |
| 3 | 1/80 | 83.7 |
| 4 | 1/160 | 57.5 |
| 5 | 1/320 | 31.7 |
| 6 | 1/640 | 0 |

A vaccine may be produced according to the procedures outlined in Example II by first exposing the same or a different mammalian species to a virus such as SIV or HIV. The preferred non-human mammal is a goat. The resultant neutralizing antibody or antibodies are extracted from the animal's serum or milk via standard methods such as ammonium sulfate or sodium sulfate precipitation and centrifugation methodology followed by purification by such methods as dialysis or gel filtration. Tijssen, P., “Practice and theory of enzyme immunoassays,” Laboratory Techniques in Biochemistry and Molecular Biology, R.H. Burdon and P.H. van Knippenberg (eds.), Amsterdam: Elsevier Science Publishers, vol. 15, pp. 96-98 (1985).

Once the neutralizing antibodies are isolated, placing the neutralizing antibody or antibodies in the presence of live SIV and/or HIV viruses (Brooks, et al., Medical Microbiology, 19^{th} ed., Appleton & Lange, East Norwalk, CN, p. 150 (1991)) or other viruses into a host species, the SIV and/or viral cells are rendered noninfectious and incapable of further replication. This process of using neutralizing antibody or antibodies to attenuate the SIV, HIV or other virus is referred to as an antibody attenuation of a virus to produce a vaccine, or “AAV2”.

### EXAMPLE III

### Preparation and Use of a Vaccine

Neutralizing antibody or antibodies are used to attenuate the SIV, HIV, and other viruses to produce a vaccine. The HIV virus is extracted from a human donor who has been diagnosed as being HIV+ and whose HIV virus is both isolated and sero-typed. (Bobkov, et al., “Identification of an env G subtype and heterogeneity of HIV- 1 strains in the Russian Federation and Belarus,” AIDS 8:1649-1655 (1994); Gao, et al., “Genetic Variation of HIV type 1 in Four World Health Organization-sponsored Vaccine Evaluation Sites: Generation of Functional Envelope (Glycoprotein 160) Clones representative of sequence subtypes A, B, C, and E, “AIDS Res Hum Retroviruses 10:1359-1368 (1995); WHO Network for HIV Isolation and Characterization, “HIV Type 1 Variation in World Health Organization-sponsored Vaccine Evaluation Sites: Genetic Screening, Sequence Analysis, and Preliminary Biological Characterization of Selected Viral Strains,” AIDS Res. Hum. Retroviruses 10:1327-1343 (1994); Delwart, et al., “Genetic relationships determined by a DNA heteroduplex mobility assay: analysis of HIV-1 env genes,” Science 262:1257-1261 (1993).

The HIV virus of the human donor is then placed on or injected into a non-human mammal such as a goat. The injection into the goat produces an immunological response in the form of neutralizing antibodies. The neutralizing antibodies are extracted from the mammal's serum or milk and then mixed with the heat killed human donor's HIV sero-typed virus to produce the attenuated antibody viral vaccine, or AAV2.

The proportion of virus to antibody requires the antibody is bound to at least one epitope on each virus particle but that not all epitopes are bound. It is believed that attachment of a single neutralizing antibody to a single epitope is sufficient to prevent fusion, and thus, virulence is removed while immunogenicity is maintained. For a given virus-neutralizing antibody(ies) combination, the neutralization assay described in Example II can be performed to determine what ratio of neutralizing antibody to virus results in complete neutralization of the virus. A vaccine is then prepared using a neutralizing antibody concentration of less than that which resulted in complete neutralization as determined in the neutralization assay.

The AAV2 is in the form of an HIV-IGA complex which maintains immunogenicity but neutralizes virulence. The AAV2 is then returned, typically by injection, back into the human donor. Placement of the AAV2 into the human donor produces a competent immunological response which blocks, prevents or destroys further progress or development of the SIV and/or HIV virus into the AIDS complex disease. It is noted that IgA and other antibodies are mixed with the human donor virus in an effective amount so as to allow immunogenicity to be attained and maintained by bonding some but not all viral epitopes, thereby yielding an immunological response different from the parent virus but still achieving neutralization of the SIV/HIV virus by rendering it an attenuated noninfectious virus (AAV2). The binding of epitopes will activate complement and virtually destroy much of the viral complex. This process yields a vaccine, or AAV2, which, when placed into a human body, denies viral replication thereby allowing the human body's immune system to be stimulated to increase its own production of competent antibodies as a result of AAV2 stimulation. This increase of the human body's competent antibodies then prevents the additional production or replication of the simian SIV or human HIV virus and the development of the AIDS complex disease.

Modern vaccination technologies are also well known in the art. For example, recombinant DNA vaccines have been produced for viruses such as cholera, herpes simplex, HIV and hepatitis B. These vaccines provide selected immunogenic peptides which can also be coupled with adjuvants. Immunogenic virus-like particles (VLP) comprising proteins of the same size and structure as viruses but without the viral DNA or RNA can be produced by fusing foreign genes to the Ty gene is yeast.

Methodologies for producing vaccines of chemically synthesized peptides which comprise the primary structure of antigenic regions of an infectious virus are also available. Also know in the art are idiotypic vaccines prepared by exposing a host to a classical antigen, allowing the host to produce antibodies (Ab-1) to the antigen, then allowing the host to produce antibodies (Ab-2) having antigenic determinants resembling the original antigen and demonstrating improved immunogenicity by maintaining the tertiary structure of the antigen. (Coleman, et al, Fundamental Immunology, 2d ed., Wm C. Brown Publishers, 1992; Keeton, W. T. and Gould, J. L., Biological Science, W. W. Norton, 1993; Solomon, et al., Biology, 3d ed., Saunders College Publishing, Harcourt Brace Jovanovich College Publishers, 1983. Recombinant DNA vaccines, VLP's, synthetic peptides, or idiotypic vaccines, rather than live virus, may be used in the present invention to produce neutralizing antibodies in a host mammal.

Synthetic peptides derived from the complementarity-determining region (CDR) sequences of antibodies may be mass-produced in vitro, which are similar to the intact antibody, inhibitory to idiotype-anti-idiotype interactions, bind specific antigens, interact with cellular receptors and stimulate biological processes. (Taub, et al., J. Biol. Chem. 264:259 (1989); Bruck, et al., Proc Natl Acad Sci USA 83:6578 (1986) Kang, et al., Science 240:1034 (1988); Williams, et al., Proc. Nat'l Acad. Sci. USA 86:5537 (1989); Novotny, et al., J. Mol. Biol. 189:715 (1986). Although these peptide analogs seem to have a limited use in vivo due to their proteinaceous characteristics such as water insolubility, high immunogenicity, their ability to adopt various conformations, and subjectability to proteolysis, it is foreseen that such synthetic peptides might be used in the present invention.

It is well known in the art that a monoclonal antibody can be mass-produced via hybridoma technology for the purpose of economically providing large amounts of a vaccine. Virus neutralizing agents in the form of monoclonal antibodies may also be used in the present invention. In this technique, neutralizing antibody producing cells are fused with immortal cells of a myeloma to produce the hybridoma cells; the hybridoma cells are screened for antibody production; the cells which produce the desired monoclonal antibody are then either cultured in large numbers in tissue culture or reinjected into the peritoneal cavities of many mice where the cells multiply and produce large quantities of monoclonal antibody in the ascites fluid that is formed; the ascites fluid containing the monoclonal antibody is collected; and the monoclonal antibody is purified by techniques such as affinity chromatography or column chromatography.

Techniques for producing non-peptide compounds referred to as mimetics have been developed which permit the synthesis of a conformationally restricted molecule that mimics the binding and functional properties of monoclonal antibodies. The mimetics are synthesized by first determining the relevant contract residues and conformation involved in the antibody-antigen binding (Williams, et al., J Biol Chem 266:5182 (1991); Segal, et al., Proc Natl Acad Sci USA 71:4298 (1974); Amzel, et al., Proc Natl Acad Sci USA 71:1427 (1974), de la Paz, et al., EMBO J 5:415 (1986); Kieber-Emmons, et al., Int Rev Immunol 2:339 (1987) and then synthesizing conformationally restricted cyclic organic peptides which have the required contact residues and conformation (Kahn, et al., J Mol Recognition 1:75 (1988); Kahn, et al., J Am Chem Soc 110:1638 (1988). Such mimetics can be prepared from the neutralizing antibodies produced by the mammalian hosts of the present invention and utilized in treatment of viral infections.

Furthermore, an isolated neutralizing antibody can be mass-produced by biomolecular sequencing techniques. The neutralizing antibody is first sequenced, and the sequence is then used as a template for in vitro production.

### EXAMPLE IV

### Immunological Barrier And Prevenatives

An immunological barrier to the transmission or invasion of SIV, HIV or other similar viruses via the mucosal surfaces may be prepared using milk and/or serum neutralizing antibodies extracted as described in Example II. These antibodies are developed into a passive immunological barrier in the form of vaginal creams, rectal creams, eye drops, oral sprays, swabs or injections to be applied directly to the mucosa or at the site of accidental needle pricks or sticks. It should be noted that with oral, vaginal or rectal sex, a latex condom should be used concurrently to achieve maximum benefit but it is not necessary if sufficient cream or spray is used. The serum from the mammal is treated with ammonium sulfate to precipitate the neutralizing antibodies. Then, the antibodies will be dissolved in an isotonic solution. Once dissolved, the solution is maintained at or less than 4 degrees Centigrade until used. Long term storage, i.e., in excess of thirty days, can be achieved at temperatures of −20 deg C.

Thereafter, sprays, gels, creams, drops and similar applications can be manufactured using standard, acceptable industrial suspension and preservation technology. Condom use can be either by manual application of the preparation to the interior of the condom prior to its use or by pre-application to the condom before packaging. Concurrent use of the preparation in the form of a vaginal cream or spray is recommended to enhance the barrier protection.

The average number of vital particles per milliliter of bodily secretions for which the barrier is intended can be determined by measuring the number for a particular patient, or for a population of patients. Once the average number is determined, then a theoretical neutralization number can be obtained using Table 4.

A human serum or a serum complex composed of neutralizing antibodies obtained from a mammal similar to, but not limited to, that described herein to arrest further cellular or viral development within the human body as set out may be developed for and against a specific SIV, HIV and/or other viruses, or mutated strain(s) of these viruses. The serum development is applicable for humans and other species. The mammal' neutralizing antibodies which have been produced as described in Example II and which have been processed and stored in solution as described in Example III can be introduced into humans who have the HIV virus or other viruses under treatment. The serum is introduced, in one form, intraveneously, intramuscularly, intradermally or subcutaneously.

### EXAMPLE V

### Preparation and Use of Serum or Serum Complex

A mammal's neutralizing antibodies, which have been produced, precipitated and stored in solution as described in Examples II and III above, can be introduced into humans who have the HIV virus or other viruses under treatment. The serum is preferably introduced subcutaneously or IM, but may be given intravenously with precaution.

### Vaccines

The present invention contemplates vaccines for use in both active and passive immunization embodiments. Immunogenic compositions, proposed to be suitable for use as a vaccine, may be prepared most readily directly from immunogenic heterologous antibody-antigen complex prepared in a manner disclosed herein. Preferably the antigenic material is extensively dialyzed to remove undesired small molecular weight molecules and/or lyophilized for more ready formulation into a desired vehicle.

The preparation of vaccines that contain the heterologous antibody-antigen as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such vaccines are prepared as injectables. Either as liquid solutions or suspensions: solid forms suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. Examples of such pharmaceutically acceptable carrier or diluents include water, phosphate buffered saline or sodium bicarbonate buffer. A number of other acceptable carriers or diluents are known.

In addition, if desired, the vaccine may contain minor amounts of auxiliary substances such as wetting or emulsifying agents, pH buffering agents or adjuvants that enhance the effectiveness of the vaccines.

Vaccines may be conventionally administered parenterraly, by injection of the heterologous antibody-antigen, for example, either subcutaneously of intramuscularly. Additional formulations which are suitable for other modes of administration include suppositories and, in some cases, oral formulations. For suppositories, traditional binders and carriers may include, for example, polyalkalene glycols or triglycerides. Such suppositories may be formed from mixtures containing the active ingredient in the range of 0.5% to 10%, preferably 1-2%. Oral formulations include such normally employed excipients as, for example, pharmaceutical grades of mannitol, lactose, starge, magnesium stearate, sodium saccharine, cellulose, magnesium carbonate and the like. These compositions take the form of solutions, suspensions, tablets, pills, capsules, sustained release formulations or powders and contain 10-95% of active ingredient, preferably 25-70%.

The heterologous antibody-antigen complex may be formulated into the vaccine as neutral or salt forms. Pharmaceutically acceptable salts, include the acid addition salts (formed with the heterologous antibody-antigen) and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine and the like.

The vaccines are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. Suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydrozide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° to 101° C. for 30 seconds to 2 minute periods, respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six vaccinations, more usually not exceeding four vaccinations and preferably one or more, usually at least about three vaccinations. The vaccinations will normally be at least from two to twelve week intervals, more usually from three to five week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionucleotides, enzymes, fluorescence, and the like. These techniques are well-known and may be found in a wide variety of patents, such as U.S. Pat. Nos. 3,791,932; 4,174,384; and 3,949,064, as illustrative of these types of assays.

### EXAMPLE VI

### Agricultural Uses

A cure or arresting mechanism to the caprine encephalitis (CAE) virus is now disclosed. The principles described herein and the processes set out in Examples II and III are also equally applicable to the CAE virus which presently affects goats or other virulent viral systems in the same manner that the SIV and HIV viruses do humans. The mammal of choice for anti-body production is the milk-producing cow. Use of these principles and techniques set forth above will result in a program that should on proper application eradicate the CAE virus from the goat herds.

Another aspect of the present invention includes the development of an in vitro diagnostic procedure for AIDS that include placing a serum or another biological medium from a patient to be diagnosed in contact with at least one of the proteins or glycoproteins of SIV, HIV-1 or HIV-2, or with a viral lysate or extract, and then detecting the immunological reaction. One method for implementing the present invention includes, for example, and ELISA in which immunoenzymatic reactions or immunofluorescent materials are used to detect the presence of an immunogenic complex. The assays may be direct or indirect immunofluorescence measurements or direct or indicrect immuno-enzymatic dosages.

Therefore, the present invention also applies to labeled virus extracts regardless of whether the labeling is enzymatic, fluorescent, radioactive, etc. Such assays illustratively include: depositing specific extract quantities or quantities of the proteins of the present invention in the wells of a microtiter plate; introducing increasingly higher dilutions of the serum to be diagnosed into these wells; incubating the microtiter plate; carefully washing the mirotiter plate with a suitable buffer; introducing antibodies that are specifically labeled with human immunoglobulins into the wells of the microtiter plate, the labeling being carried out by an enzyme selected from those capable of hydrolyzing a substrate in such a manner that this substrate thereupon alters its radiation-absorptivity at least within a specific band of wavelengths; and detecting, preferably in comparative manner with respect to a control, the amount of substrate hydrolysis both with respect to measuring potential danger and any actual presence of the ailment.

another aspect of the present invention are kits for the above diagnostic procedure. These kits include: an extract or a more purified fraction of the above described virus types, where this extract or fraction is labeled, for example, radioactively, enzymatically or by immunofluorescence; human anti-immunoglobulins or a protein A (advantageously fixed on a water-insoluble support such as Agarose beads); an extract of lymphocytes obtained from a healthy person as a control; buffers, and where called for, substrates to visualize the labels.

Additionally, the vaccine or immune modulator may be used in a variety of forms to enhance the immune response of an individual that has been infected by HIV in the following forms:

I. As a topical cream against HIV associate Kaposi's sarcoma.

II. In an intravenously solution such as saline may be effective in reducing viral load and slowing down the onset of immunodeficiency. Surgeons who also use saline washes in cleansing a particular area in the operating field may find it useful. The use of the heterologous antibody-antigen as well as liposomalization may be specifically included. These forms could be reconstituted in the form of mouthwash with the heterologous antibody-antigen alone or in conjunction with antifungal reagents. An inhalant form alone or in conjunction with pentamidine. The use of the heterologous antibody-antigen in tablet form to be taken orally.

III. Buffer ophthalmic solution—for patients suffering from HIV associated retinitis. The buffering is necessary due to pH changes the heterologous antibody-antigen may cause.

IV. Highly concentrated solution for intramuscular injection—would facilitate treatment of needle stick injuries of health care workers. In this regard, use of the DMSO as solvent would give extremely fast penetration delivering high concentrations of heterologous antibody-antigen to a small area.

V. Suppository form—for chemoprevention in homosexuals because the major sites of infection are the large intestine and rectum.

VI. Chemo-preventative Vaginal douche and creme—the douche may be of use in a pre-sexual exposure in a standard acetic acid solution. The creme may be mixed with 9—nonoxynol spermicide to use in conjunction with birth control.

VII. The creme described in VI could also be used in condoms.

VIII. Vaginal sponge—this could be used by prostitutes so that heterologous antibody-antigen would be time-released over several hour with nonoxynol-9.

IX. Gloves lined with heterologous antibody-antigen may help surgeons and other health care workers dealing heavily with blood and bodily fluids.

X. The use of heterologous antibody-antigen in liquid soap in combination with anti-bacterial agents may be useful in hospitals and research institutions. Although this would probably be no more effective than plain anti-bacterial soap, the employees and hospital insurance companies would appreciate it.

XI. The attachment of heterologous antibodies to sterile HPLC resin (for example SiO₃-peptide or Si (peptide)₄, etc.) to be used as a disposable filter for blood and blood by-products prior to patient transfusion. This is to insure that the donor is not in the seroconversion window.

Acqueous compositions (inocula) of the heterologous antibody solution as described herein, and include an effective amount of the heterologous antibody-antigen complex dissolved or dispersed in a pharmaceutically acceptable aqueous medium. As used herein, the terms “contact”, “contacted”, and “contacting”, are used to describe the process by which an effective amount of a pharmacological agent, e.g., pure or dilute heterologous antibody-antigen complex, comes is direct juxtaposition with the target cell. The phrase “pharmaceutically acceptable” refers to molecular entities and compositions that do not produce an allergic or similar untoward reaction when administered to a human, such as the heterologous antibody-antigen as described herein.

The preparation of an acqueous composition that contains a protein or proteoglycan, such as the active components derived from heterologous antibody-antigen complexes, is well understood in the art. Typically, such compositions are prepared as injectables, either as liquid solutions or suspensions; solid forms suitable for solution in, or suspension in, liquid prior to injection can also be prepared. The preparation can also be emulsified.

Proteoglycans, for example, may be formulated into a compositions in a neutral or salt form. Pharmaceutically acceptable salts, include the acid addition salts (formed with the free amino groups of the protein) and are formed with inorganic acids such as, for example, hydrochloric or phosphoric acids, or such organic acids as acetic, oxalic, tartaric, mandelic and the like. Salts formed with the free carboxyl groups may also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like.

For parenteral administration in an aqueous solution, for example, the heterologous antibody-antigen complex may be used directly without any toxic effects to the animal. Alternatively, the heterologous antibody-antigen complex and carrier solution may be dissolved or resuspended, in a suitable buffer, if necessary. Liquid diluents can first be rendered isotonic with sufficient saline or glucose.

While these particular acqueous solutions are especially suitable for intravenous, intramuscular, subcutaneous and intraperitoneal administration, the heterologous antibody-antigen carrier solution of the present invention can be administered directly at full concentration. In this connection, sterile acqueous procedures to produce heterologous antibody-antigen carrier may be employed, as will be known to those skilled in the art in light of the present disclosure. For example, one dosage could be dissolved in 1 mL of isotonic NaCI solution and either added to 1000 mL of hypodermoclysis fluid or injected at the proposed site of infusion, (see for example, “Remington's Pharameutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

While this invention has been described in reference to illustrative embodiments, this description is not intended to be construed in a limiting sense. Various modifications and combinations of the illustrative embodiments, as well as, other embodiments of the invention, will be apparant to persons skilled in the art upon reference to the description. It is, therefore, intended that the appended claims encompass any such modifications or embodiments.

What is claimed is:

1. An immunomodulatory composition comprising:
heterologous antibodies specific for an antigen; and
an antigen, wherein the heterologous antibodies form a complex with the antigen for combination with a pharmaceutically acceptable carrier.

2. The composition of claim 1, further comprising a pharmaceutically acceptable carrier.

3. The composition of claim 1, wherein the heterologous antibodies are polyclonal.

4. The composition of claim 1, wherein the heterologous antibodies are monoclonal.

5. The composition of claim 1, wherein the heterologous antibodies are raised in a goat.

6. The composition of claim 1, wherein the heterologous antibodies are goat anti-retrovirus antibodies.

7. The composition of claim 1, wherein the heterologous antibodies are polyclonal antibodies raised in a pregnant goat and are isolated from goat milk.

8. The composition of claim 1, wherein the antigen is a heat killed human immunosuppressive virus.

9. The composition of claim 1, wherein the antigen is a heat killed simian immunosuppressive virus.

10. The composition of claim 1, wherein the antigen is a chemically inactivated immunosuppressive pathogen.

11. The composition of claim 1, wherein the carrier is buffered saline.

12. The composition of claim 1, wherein the antigen is an attenuated immunosuppressive pathogen.

13. The composition of claim 1, wherein the antigen is isolated from the patient to be treated with the composition and the heterologous antibodies are raised against that specific isolate.

14. A vaccine for stimulating immune responses in an immunosuppressed host comprising:
heterologous antibodies specific for an antigen;
an antigen, wherein the heterologous antibodies form a complex with the antigen; and
a pharmaceutically acceptable carrier.

15. The composition of claim 14, further comprising a pharmaceutically acceptable carrier.

16. The composition of claim 14, wherein the heterologous antibodies are polyclonal.

17. The composition of claim 14, wherein the heterologous antibodies are monoclonal.

18. The composition of claim 14, wherein the heterologous antibodies are raised in a goat.

19. The composition of claim 14, wherein the heterologous antibodies are goat anti-retrovirus antibodies.

20. The composition of claim 14, wherein the heterologous antibodies are polyclonal antibodies raised in a pregnant goat and are isolated from goat milk.

21. The composition of claim 14, wherein the antigen is a heat killed human immunosuppressive virus.

22. The composition of claim 14, wherein the antigen is a heat killed simian immunosuppressive virus.

23. The composition of claim 14, wherein the antigen is a chemically inactivated immunosuppressive pathogen.

24. The composition of claim 14, wherein the carrier is buffered saline.

25. The composition of claim 14, wherein the antigen is an attenuated immunosuppressive pathogen.

26. The composition of claim 14, wherein the antigen is isolated from the patient to be treated with the composition and the heterologous antibodies are raised against that specific isolate.

27. A method of vaccinating a host against an immunosuppressive pathogen, which method comprises administering to said host an effective amount of an attenuated antigen from an immunosuppressive pathogen and heterologous antibodies as defined in claim 1.

28. The method of claim 27, wherein the heterologous antibodies are polyclonal.

29. The method of claim 27, wherein the heterologous antibodies are monoclonal.

30. The method of claim 27, wherein the heterologous antibodies are raised in a goat.

31. The method of claim 27, wherein the heterologous antibodies are goat anti-retrovirus antibodies.

32. The method of claim 27, wherein the heterologous antibodies are polyclonal antibodies raised in a pregnant goat and are isolated from goat milk.

33. The method of claim 28, wherein the antigen is a heat killed human immunosuppressive virus.

34. The method of claim 29, wherein the antigen is a heat killed simian immunosuppressive virus.

35. The method of claim 29, wherein the antigen is a chemically inactivated immunosuppressive pathogen.

36. The method of claim 29, wherein the carrier is buffered saline.

37. The method of claim 29, wherein the antigen is an attenuated immunosuppressive pathogen.

38. The method of claim 29, wherein the antigen is isolated from the patient to be treated with the composition and the heterologous antibodies are raised against that specific isolate.

39. A method of immunomodulation comprising the steps of:
isolating an immunosuppressive etiologic agent from an immune suppressed host;
purifying the immunosuppressive etiologic agent;
inoculating an animal across species barriers with the purified immunosuppressive etiologic agent;
purifying xenotypic antibodies from the animal specific to the immunosuppressive etiologic agent; and
mixing inactivated immunosuppressive etiologic agent with the purified xenotypic antibodies to produce an inoculant.

40. The method of claim 39, further comprising the step of inoculating the immune suppressed host with the inoculant.

41. The method of claim 39, wherein the heterologous antibodies are polyclonal.

42. The method of claim 39, wherein the heterologous antibodies are raised in a goat.

43. The method of claim 39, wherein the heterologous antibodies are goat anti-retrovirus antibodies.

44. The method of claim 39, wherein the heterologous antibodies are polyclonal antibodies raised in a pregnant goat and are isolated from goat milk.

45. The method of claim 39, wherein the immunosuppressive etiologic agent is a heat killed human immunosuppressive virus.

46. The method of claim 39, wherein the immunosuppressive etiologic agent is a heat killed simian immunosuppressive virus.

47. The method of claim 39, wherein the immunosuppressive etiologic agent is a chemically inactivated immunosuppressive pathogen.
